(19) United States
(12) Patent Application Publication
(10) Pub. No.: US 2004/0171811 A1
(43) Pub. Date: Sep. 2, 2004

(54) Nonhuman pregnane x receptor sequences for use in comparative pharmacology

(76) Inventors: Steven Anthony Kliewer, Dallas, TX (US); Jodi Marie Maglich, Durham, NC (US); John Tomlin Moore, Durham, NC (US); Linda Becker Moore, Durham, NC (US); Timothy Mark Willson, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/478,551

(22) PCT Filed: May 24, 2002
(86) PCT No.: PCT/US02/16445

Related U.S. Application Data
(60) Provisional application No. 60/293,380, filed on May 24, 2001.

(51) Int. Cl. C07K 14/72; C12Q 1/68; C07H 21/04

(52) U.S. Cl. 530/358; 435/6; 435/69.1; 435/320.1; 435/325; 536/23.5

(57) ABSTRACT

Polynucleotides and polypeptides of canine, primate, porcine and fish pregnane X receptor (PXR), as well as expression vectors and host cells for expression of these PXR receptors, are provided. Also provided are methods for screening for modulators of these PXR receptors and using these receptors for comparative pharmacology and in selection of appropriate preclinical animal models predictive of human PXR activity.

### FIELD OF THE INVENTION

The present invention relates to nonhuman pregnane X receptor (PXR) polynucleotide and polypeptide sequences isolated from fish, canines, porcine and primates and their use in comparative pharmacology.

### BACKGROUND OF THE INVENTION

Members of the cytochrome P450 family of hemoproteins are critical in the oxidative metabolism of a wide variety of endogenous substances and xenobiotics, including various carcinogens and toxins (Nebert et al. (1987) Ann. Rev. Biochem. 56:945-993). In man, P450 3A4 monooxygenase, also referred to as CYP3A4 monooxygenase, plays a major role in the biotransformation of drugs due to its abundance in liver and intestine and its broad substrate specificity. P450 3A4 catalyzes the metabolism of >60% of all drugs that are in use including steroids, immunosuppressive agents imidazole antimycotics, and macrolide antibiotics (Maurel, P. in Cytochrome P450: metabolic and toxicological aspects (ed. Ioannides, C.) 241-270 (CRC Press, Inc. Boca Raton, Fla. 1996).

Expression of. P450 3A4 is induced both in vivo and in primary hepatocytes in response to treatment with a variety of compounds including, but not limited to, commonly used drugs such as the glucocorticoid dexamethasone, the antibiotic rifampicin, the antimycotic clotrimazole, and the hypocholesterolemic agent lovestatin (Maurel, P. in Cytochrome P450: metabolic and toxicological aspects (ed. Ioannides, C.) 241-270 (CRC Press, Inc. Boca Raton, Fla. 1996); Guzelian, P. S. in Microsomes and Drug Oxidation (eds. Miners, J. O., Birkett, D. J., Drew, R. & McManus, M.) 148-155 (Taylor and Francis, London, 1988)). The inducibility of P450 3A4 expression levels coupled with its broad substrate specificity represent the basis for many drug interactions in patients undergoing combination therapy. Thus, analysis of the effects of new compounds on P450 3A4 gene expression is an important aspect in drug development.

While attempts have been made to develop in vivo and in vitro assays with which to profile the effects of compounds on P450 3A4 expression levels, poorly understood species-specific variations have limited the utility of using animals and their tissues for testing purposes. Thus, analysis of the effects of new compounds on P450 3A4 gene expression has been largely restricted to laborious assays involving human liver tissue.

Nuclear hormone receptors comprise a large superfamily of ligand-modulated transcription factors that, in part, mediate responses to steroids, retinoids, and thyroid hormones (for review see Beato et al., (1995) Cell 83:851-857; Kastner et al., (1995) Cell 83:859-869; Mangelsdorf and Evans, (1995) Cell 83:841-850). Detailed analysis of the receptors, most notably the steroid class of receptors, has revealed multiple discrete functional modules within the family that display generalized functional characteristics (Tzukerman et al., (1994) Mol. Endocrinol. 8:21-30). A variable amino-terminal domain (A/B) is present that typically contains a strong and autonomous activation function (AF1), shown to be critical for cell and target gene specificity (Tora et al., (1988) Nature 333: 677-684). A more carboxyl-terminal central region contains a DNA binding domain (DBD) characterized by two C4-type zinc fingers. The DBD binds to specific genomic response elements and thereby regulates the transcriptional activity of select genes containing the response elements. At the distal carboxyl terminus, a ligand binding domain (LBD) is present containing a highly conserved second transactivation function (AF2) that is important for hormone-dependent transcriptional transactivation (Lanz and Rusconi, (1994) Endocrinology 135: 2183-2195). Sequences that function in nuclear localization, receptor dimerization, and interaction with heat-shock proteins (Gronemeyer and Laudet, (1995) CCQ 2:1173-1308) are also present within the nuclear receptor substructure. Through the coordinated action of these separate functional domains, nuclear receptor activation by ligand culminates in modulation of target gene expression (Tsai and O'Malley, (1994) Ann. Rev. Biochem. 63: 451-486) and in certain cases, cross-talks with other cell signaling pathways such as the NF-kB (Stein and Yang, (1995) Mol. Cell. Biol. 15: 4971-4979) and AP-1 (Paech et al., (1998) Science 277: 1508-1510). Ultimately, ligand alters nuclear receptor function by altering the constellation of protein-protein interactions in which the receptor is engaged (for review, see Freedman, (1999) Cell 97: 5-8). The molecular details underlying the multitude of cellular effects mediated by nuclear receptors are the subject of intense research activity.

For the nuclear receptor for pregnane X, referred to herein as PXR (Unified Nomenclature Committee designation NR1I2), it has been shown that PXR cell-based and binding assays are predictive of in vivo effects on the cytochrome P450 3A4 gene (Kliewer et al. (1998) Cell 92, 73-8273-82;

Lehmann et al. (1998) J. Clin. Invest. 102, 1016-1023; Jones et al. (2000) Mol. Endo. 27-39).

WO 99/48915 discloses human PXR which binds to the CYP promoter rifampicin/dexamethasone response element in cytochrome P450 3A4. Also disclosed are nucleic acid sequences encoding human PXR, as well as vectors and host cells for expression of the human receptor, and methods for using this receptor in vitro to screen compounds for their ability to modulate P450 3A4 expression in humans.

However, nonhuman animal PXRs, particularly animals well accepted for use in preclinical studies, would also be useful in the development in vitro and in vivo animal models 35 for profiling the effects of compounds on P450 3A4 expression levels and to select preclinical models predictive of effects in humans. Additionally, dissecting the broader biological hysiological role of PXR beyond cytochrome P450 gene induction is facilitated by an understanding of which compounds activate PXR in animal models of interest. Thus, comparative pharmacology of PXR opens the possibility of extending the utility of PXR in drug development and toxicity assays as well as validating this receptor as a useful target in disease.

Mouse PXR has been cloned and sequenced (Kliewer et al. (1998) Cell 92:73-82). Rat and rabbit PXR have also been cloned and sequenced (Jones et al. (2000) Molecular Endocrinology 14(l):27-39; Zhang et al. (1999) Archives of Biochemistry and Biophysics 368(1):14-22).

The present invention relates to PXRs for other nonhuman animals.

### SUMMARY OF THE INVENTION

Polynucleotide and polypeptide sequences for pregnane X receptors (PXR) isolated from fish, canines, porcine and primates are provided. The novel PXR sequences are useful as screening targets for the identification and development of selective PXR compounds. These agents are particularly useful in PXR comparative pharmacology and selecting appropriate animal models for preclinical studies predictive of effects in humans.

Accordingly, the present invention provides isolated PXR polypeptides comprising:

(a) an amino acid sequence of SEQ ID NO: 2, 4, 6 or 8;

(b) a variant of an amino acid sequence as defined in (a) which modulates P450 3A4 levels or activity; or

(c) a fragment of (a) or (b) which modulates P450 3A4 levels or activity.

According to another aspect of the invention there is provided polynucleotides encoding polypeptides of the invention, said polynucleotides comprising:

(a) a nucleic acid sequence of SEQ ID NO: 1, 3, 5, or 7;

(b) a nucleic acid sequence which hybridizes under stringent conditions to the nucleic acid sequence as defined in (a);

(c) a nucleic acid sequence that is degenerate as a result of the genetic code to the nucleic acid sequences as defined in (a) or (b); or

(d) a nucleic acid sequence having at least 60% identity to the nucleic acid sequences as defined in (a), (b) or (c).

Other related aspects of the present invention include expression vectors comprising polynucleotides of the invention which are capable of expressing a polypeptide of the invention, host cells comprising an expression vector of the invention, methods of producing a polypeptide of the invention which comprise maintaining a host cell of the invention under conditions suitable for obtaining expression of the polypeptide and isolating said polypeptide, antibodies specific for a polypeptide of the invention, and transgenic nonhuman animals expressing a mutant PXR or a PXR from another species.

The present invention also provides methods for identification of substances that modulate PXR activity and/or expression. In one embodiment, the method comprises contacting a polypeptide, polynucleotide, expression vector or host cell of the invention with a test substance and determining the effect of the test substance on the activity and/or expression of the polypeptide to determine whether the test substance modulates PXR activity and/or expression. In another embodiment, the test substance is administered to a nonhuman transgenic animal expressing a mutant PXR or a PXR from a different species and the effects of the test substance on expression and/or activity of the receptor are examined.

In addition, the present invention relates to compounds which modulate PXR activity and which are identifiable by the methods referred to above.

### BRIEF DESCRIPTION OF THE SEQUENCES

SEQ ID NO: 1 shows a nucleotide and amino acid sequence of a ligand binding domain of a canine pregnane X receptor.

SEQ ID NO: 2 shows the amino acid sequence of the ligand binding domain of the canine pregnane X receptor as depicted in SEQ ID NO:1.

SEQ ID NO:3 shows a nucleotide and amino acid sequence of a primate pregnane X receptor.

SEQ ID NO:4 shows the amino acid sequence of the primate pregnane X receptor as depicted in SEQ ID NO:3.

SEQ ID NO:5 shows a nucleotide and amino acid sequence of a ligand binding domain of a porcine pregnane X receptor.

SEQ ID NO:6 shows the amino acid sequence of the ligand binding domain of the porcine pregnane X receptor as depicted in SEQ ID NO:5.

SEQ ID NO:7 shows a nucleotide and amino acid sequence of a ligand binding domain of a Zebrafish pregnane X receptor.

SEQ ID NO:8 shows the amino acid sequence of the ligand binding domain of the Zebrafish pregnane X receptor as depicted in SEQ ID NO:7.

### BRIEF DESCRIPTION OF THE FIGURES

FIG. 1 is a bargraph showing the activation of a primate PXR in the presence of various steroids and xenobiotics.

FIG. 2 is a bargraph showing the activation of a canine PXR in the presence of various steroids and xenobiotics.

FIG. 3 is a bargraph showing the activation of a porcine PXR in the presence of various steroids and xenobiotics.

FIG. 4 is a bargraph showing the activation of a fish PXR in the presence of various steroids and xenobiotics.

FIG. 5 is a bargraph showing the activation of a primate PXR in the presence of various bile acids.

FIG. 6 is a bargraph showing the activation of a canine PXR in the presence of various bile acids.

FIG. 7 is a bargraph showing the activation of a porcine PXR in the presence of various bile acids.

FIG. 8 is a bargraph showing the activation of a fish PXR in the presence of various bile acids.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to nonhuman animal orthologs of pregnane X receptors, referred to herein as PXRs, and variants thereof. More specifically, the present invention relates to PXRs isolated from a canine, porcine, primate or fish. Nucleotide sequence information for the full length monkey PXR of the present invention is provided in SEQ ID NO: 3. A polypeptide sequence of the monkey PXR is also provided in SEQ ID NO: 4. Sequence information for the ligand binding domains of the dog, pig and Zebrafish PXRs of the present invention is provided in SEQ ID NO: 1, 5 and 7 (nucleotide and amino acid), respectively. Polypeptides of the ligand binding domains of the dog, pig and Zebrafish PXRs are also provided in SEQ ID NO: 2, 6, and 8.

Polypeptides of the invention consist essentially of the amino acid sequences of SEQ ID NO: 2, 4, 6 or 8, a variant of that sequence, or a fragment of either thereof. Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesize the polypeptides according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The term “variant” refers to a polypeptide that has a same essential character or basic biological functionality as the selected PXR. As demonstrated herein, biological functionalities of nonhuman PXRs vary between species. By “selected PXR receptor”, as used herein, it is meant a canine, primate, porcine or fish PXR as described herein. Further, for purposes of this invention a variant polypeptide is preferably one which binds to the same ligand as one or more of the nonhuman animal PXRs described herein. Preferably the polypeptide modulates P450 3A4 expression in primates, canines, porcine and/or fish. A polypeptide having a same essential character as a selected PXR of the present invention can be identified by monitoring for activation of the selected PXR by an inducer of P450 3A4. A full-length variant polypeptide is preferably one which includes the entire ligand binding domain of the selected PXR.

In another aspect of the invention, a variant is one which does not show the same activity as the selected PXR but rather inhibits a basic function of the PXR. For example, a variant polypeptide is one which inhibits activation of a selected PXR upon exposure to an inducer of P450 3A4, for example by binding to a PXR ligand to prevent activity mediated by ligand binding to the selected PXR.

Typically, polypeptides with more than about 65% identity preferably at least 80% , at least 85% or at least 90% and particularly preferably at least 95%, at least 97% or at least 99% identity, with the amino acid sequences of SEQ ID NO: 2, 4, 6 or 8 are considered as variants of the proteins. Identity can be determined using a program such as a BLAST sequence alignment program. Such variants may include allelic variants and the deletion, modification or addition of single amino acids or groups of amino acids within the protein sequence, as long as the peptide maintains a basic biological functionality of the selected PXR.

Amino acid substitutions may be made, for example from 1, 2 or 3 to 10, 15, 20 or 30 substitutions. The modified polypeptide generally retains the same activity as a selected PXR. Conservative substitutions may be made, for example according to the following Table 1. Amino acids in the same block in the second column and preferably in the same line in the third column may be substituted for each other.

TABLE 1

| | | |
|---|---|---|
| ALIPHATIC | Non-polar | G A P |
| | | I L V |
| | polar-uncharged | C S T M |
| | | N Q |
| | Polar-charged | D E |
| | | K R |
| AROMATIC | | H F W Y |

Exemplary substitutions are shown in Table 2.

TABLE 2

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Shorter polypeptide sequences, also referred to herein as “fragments” are within the scope of the invention. For example, a peptide of at least 15, 20 or 30 amino acids or up to 50, 60, 70, 80, 100, 150 or 200 amino acids in length is considered to fall within the scope of the invention as long as it demonstrates a basic biological functionality of a selected PXR. In particular, but not exclusively, this aspect of the invention encompasses the situation when the protein is a fragment of the complete protein sequence and may represent particularly, the A/B domain (e.g., about amino acids 1-40 of SEQ ID NO:4), the DBD (e.g., about amino acids 41-105 of SEQ ID NO:4) or the LBD (e.g., about amino acids 106-434 of SEQ ID NO:4 or SEQ ID NO:2, 6 or 8), alone or in combination. Such fragments can be used to construct chimeric receptors preferably with another nuclear receptor, more preferably with another member of the family of nuclear receptors involved in P450 3A4 regulation such as the CAR nuclear receptor, or as intermediates in the production of the full length sequences.

Such fragments of PXR or a variant thereof can also be used to raise anti-PXR antibodies. In this embodiment, the fragment may comprise an epitope of a selected PXR polypeptide and may otherwise not demonstrate the ligand binding or other properties of the selected PXR.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for canine, primate, porcine or fish PXR or a variant thereof, as well as nucleotide sequences which are complementary thereto. The nucleotide sequence may be RNA or DNA including genomic DNA, synthetic DNA or cDNA. Preferably the nucleotide sequence is a DNA sequence, and most preferably a cDNA sequence. Nucleotide sequence information for the canine, primate, porcine and Zebrafish PXRs of the present invention are provided in SEQ ID NO: 1, 3, 5 and 7, respectively. Such nucleotides can be isolated from cells of the selected species, namely, canine, primate, porcine or Zebrafish, or synthesized according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of either SEQ ID NO: 1, 3, 5 or 7.

The polynucleotide can be in single-, double- or triple-stranded form. In addition, polynucleotide, as used herein, can refer to triple-stranded regions comprising RNA or DNA or both. The strands in such regions may be from the same molecule or from different molecules. As used herein the term polynucleotide includes nucleic acids that contain one or more modified (e.g., tritylated) or unusual (e.g., inosine) bases. Thus, DNAs or RNAs with backbones modified for stability or for other reasons are polynucleotides as that term is intended herein.

A polynucleotide of the invention can hybridize to the coding sequence or the complement of the coding sequence of SEQ ID NO: 1, 3, 5 or 7 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1, 3, 5 or 7 is typically at least 10-fold, preferably at least 100-fold, as intense as interactions between other polynucleotides and the coding sequence of SEQ ID NO: 1, 3, 5 or 7. The intensity of interaction may be measured, for example, by radiolabeling the probe, e.g. with ³²P. Selective hybridization may typically be achieved using conditions of medium to high stringency. However, such hybridization may be carried out under any suitable conditions known in the art (see Sambrook et al, 1989). For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 1, 3, 5 or 7 may be modified by nucleotide substitutions, for example from 1, 2 or 3 to 10, 15, 25, 50 or 100 substitutions. The polynucleotide of SEQ ID NO: 1, 3, 5 or 7 may alternatively or additionally be modified by one or more insertions and/or deletions and/or by an extension at either or both ends. A polynucleotide may include one or more introns. For example, in one embodiment, the polynucleotide may comprise genomic DNA. Additional sequences such as signal sequences which may assist in insertion of the polypeptide in a cell membrane may also be included. The modified polynucleotide generally encodes a polypeptide which has the same activity as a selected PXR. Alternatively, a polynucleotide encodes a ligand-binding portion of a polypeptide or a polypeptide which inhibits an activity of a selected PXR. Degenerate substitutions may be made and/or substitutions may be made which would result in a conservative amino acid substitution when the modified sequence is translated, for example as shown in Table 1 or 2 above.

A nucleotide sequence which is capable of selectively hybridizing to the complement of the DNA coding sequence of SEQ ID NO: 1, 3, 5, or 7 will generally have at least 60%, at least 70%, at least 80%, at least 90%, at least 95%, at least 98% or at least 99% sequence identity to the coding sequence of SEQ ID NO: 1, 3, 5 or 7 over a region of at least 20, preferably at least 30, for instance at least 40, at least 60, more preferably at least 100 contiguous nucleotides, or most preferably over the full length of SEQ ID NO: 1, 3, 5 or 7.

For example the UWGCG Package provides the BESTFIT program which can be used to calculate homology (for example used on its default settings) (Devereux et al.,(1984) Nucleic Acids Res. 12: 387-395). The PILEUP and BLAST algorithms can be used to calculate homology or line up sequences (typically on their default settings), for example as described in Altschul (1993) J. Mol. Evol. 36: 290-300; Altschul et al (1990) J. Mol. Biol. 215: 403-410.

Software for performing BLAST analyses is publicly available through the National Center for Biotechnology Information (http: //www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al. 1990). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the polypeptides according to the invention. Produciton may take place in vitro, in vivo or ex vivo. The nucleotides may be involved in recombinant protein synthesis, as therapeutic agents in their own right, utilized in gene therapy techniques, and/or utilized in the production of nonhuman transgenic animals. Nucleotides complementary to those encoding PXR, or antisense sequences, may also be used therapeutically.

Polynucleotides of the invention may be used as primers, e.g. PCR primers or primers for an alternative amplification reaction, or as probes, e.g. polynucleotides detectably labeled by conventional means using radioactive or non-radioactive labels. In addition, the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1, 3, 5 or 7.

The polynucleotides of the present invention are also useful in the production of chimeric receptors or fusion proteins having a PXR component which comprises at least a DNA binding domain or a ligand binding domain of a canine, primate, porcine or fish PXR and a non-PXR derived sequence. Non-PXR derived sequences can be selected so as to be suitable for the purpose to be served by the chimeric receptor. Examples of such sequences include, but are not limited to, glutathione-S-transferase, the DNA binding domain of yeast transcription factor GAL4 and other DNA binding domains such as the DNA binding domains for estrogen or glucocorticoid receptors, and the viral VP16 transcriptional activation domain. Chimeric receptors of the present invention may further comprise a detectable label such as a radioactive or fluorescent label. The chimeric receptors may also be bound to a solid support such as glass or plastic particles or plates or a filter.

The present invention also includes expression vectors that comprise nucleotide sequences encoding the polypeptides or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art based upon teachings provided herein and what is known in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful therapeutically.

Preferably, a polynucleotide of the invention used in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. For example, a regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with an origin of replication, optionally a promoter for the expression of the polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example, for the production of DNA or RNA or used to transfect or transform a host cell, for example, a mammalian host cell. The vectors may also be adapted to be used in vivo, for example in a method of gene therapy or in the production of nonhuman transgenic animals, preferably mice. Additional vector components known in the art are suitable for use in the vectors of the present invention and include, for example, processing sites such as a polyadenylation signal, ribosome binding sites, RNA splice sites, and transcriptional termination sequences.

Promoters and other expression regulation signals may be selected to be compatible with the host cell for which expression is designed. Examples of yeast promoters which can be used in the present invention include S. cerevisiae GAL4 and ADH promoters, and S. pombe NMT1 and ADH promoters. Viral promoters can also be used. Examples of viral promoters include, but are not limited to, the Moloney murine leukemia virus long terminal repeat (MMLV LTR), the Rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). A mammalian promoter useful in the present invention is the metallothionein promoter which can be induced in response to heavy metals such as cadmium and β-actin promoters. Tissue-specific promoters are especially preferred. All these promoters, as well as additional promoters useful in the present invention, are readily available in the art.

The vector may further include sequences flanking the polynucleotide giving rise to polynucleotides which comprise sequences homologous to eukaryotic genomic sequences, preferably mammalian genomic sequences or viral genomic sequences. This allows the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide into the host genome. Replication-defective adenovirus vectors by contrast remain episomal and therefore allow transient expression.

The invention also includes cells that have been modified to express the PXR polypeptide or a variant thereof. Such cells include transient, or preferably stable higher eukaryotic cell lines such as mammalian cells or insect cells, lower eukaryotic cells such as yeast, or prokaryotic cells such as bacterial cells. Examples of cells which may be modified by insertion of vectors encoding for a polypeptide according to the invention include, but are not limited to, mammalian HEK293T, CHO, HeLa and COS cells.

A polypeptide of the invention may also be expressed in cells of a transgenic non-human animal, preferably a mouse. Accordingly, transgenic non-human animals expressing a PXR polypeptide of the invention are also included within the scope of the invention. For example, transgenic mice can be generated that express a selected PXR of the present invention as well as the endogenous mouse PXR gene. Mice can also be generated in which the endogenous PXR gene is knocked out and then replaced by the selected PXR polynucleotide of the present invention. Transgenic animals can also be generated that express isoforms of a selected PXR as well as mutant alleles of the PXRs of the present invention. Transgenic animals developed by these methods can be used to screen compounds for drug interactions and toxicities and to study the regulation of P450 3A4 in vivo.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right.

Antibodies can be raised against specific epitopes of the polypeptides according to the invention. Such antibodies may be used to block ligand binding to the receptor. An antibody, or other compound, “specifically binds” to a protein when it binds with preferential or high affinity to the protein or polypeptide for which it is specific but does not substantially bind or binds with only low affinity to other proteins. A variety of protocols for competitive binding or immunoradiometric assays to determine the specific binding capability of an antibody are well known in the art (see for example Maddox et al, (1993) J. Exp. Med. 158: 1211-1226). Such immunoassays typically involve the formation of complexes between the specific protein and its antibody and the measurement of complex formation.

Antibodies of the invention may be antibodies to the canine, primate, porcine or fish polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragments thereof may be chimeric antibodies, CDR-grafted antibodies or humanized antibodies.

Antibodies may be used in methods for detecting polypeptides of the invention in a biological sample. In these methods, an antibody of the invention is first provided. A biological sample is then incubated with the antibody under conditions which allow for the formation of a complex between the antibody and the polypeptide or antigen and the amount of antibody-polypeptide complex formed is determined. Various methods for determining formation of an antibody-antigen complex are well known to those of skill in the art.

For purposes of the present invention, by “biological sample” it is meant to include, but is not limited to, tissue extracts, blood, serum, saliva, urine, cerebral spinal fluid, and bile.

Antibodies of the invention may be bound to a solid support and/or packaged into kits in a suitable container along with suitable reagents, controls, instructions, etc. Antibodies may be linked to a revealing label and thus may be suitable for use in methods of in vivo PXR imaging.

Antibodies of the invention can be produced by any suitable method. Means for preparing and characterizing antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunizing a suitable host animal, for example an experimental animal, with the immunogen and isolating immunoglobulins from the animal's serum. In this method, the animal is inoculated with the immunogen. Blood is subsequently removed from the animal and the IgG fraction purified.

A method for producing a monoclonal antibody comprises immortalizing cells which produce the desired antibody. Hybridoma cells can be produced by fusing spleen cells from an inoculated experimental animal with tumor cells (Kohler and Milstein (1975) Nature 256: 495-497). An immortalized cell producing the desired antibody may be selected by a conventional procedure. Hybridomas are then grown in culture or injected intraperitoneally for formation of ascites fluid or into the blood stream of an allogenic host or immunocompromised host.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

A further aspect of the present invention relates to in vitro (cell-free) and in vivo (cell-based) assays that can be used to profile the effects of compounds, particularly potential new drugs, on P450 3A4 levels in various species. These assays can take any of a variety of forms. Since compounds that activate PXR function are inducers of P450 3A4 gene expression, binding and activation assays using selected PXRs of the present invention provide efficient means to identify compounds expected to activate P450 3A4 in a selected species.

Binding assays of the present invention include cell free assays in which a selected PXR, or the ligand binding domain of a selected PXR (alone or present as a fusion protein), is incubated with a test compound which, advantageously, bears a detectable label (e.g. a radioactive or fluorescent label). The selected PXR, or ligand binding domain thereof, free or bound to the test compound, is then separated from free test compound using any of variety of techniques (e.g., using gel filtration chromatography (for example, on Sephadex G50 spin columns) or through capture on a hydroxyapatite resin). The amount of test compound bound to the selected PXR or ligand binding domain thereof, is then determined via detection of the label.

An alternative approach for detecting radiolabeled test compound bound to a selected PXR, or ligand binding domain thereof, is a scintillation proximity assay (SPA). In this assay, a bead (or other particle) is impregnated with scintillant and coated with a molecule that can capture the selected PXR, or ligand binding domain thereof (e.g., streptavidin-coated beads can be used to capture biotinylated PXR ligand binding domain). Radioactive counts are detected only when the complex of radiolabeled test compound and the selected PXR, or ligand binding domain thereof, is captured on the surface of the SPA bead bringing the radioactive label into sufficient proximity to the scintillant to emit a signal.

This approach has the advantage of not requiring the separation of free test compound from bound (Nichols et al, Anal. Biochem. 257:112-119 (1998)).

Assays to determine whether a test compound interacts with a selected PXR ligand binding domain can also be performed via a competition binding assay. In this assay, the selected PXR, or ligand binding domain thereof, is incubated with a compound known to interact with the selected PXR, which compound, advantageously, bears a detectable label (e.g., a radioactive or fluorescent label). A test compound is added to the reaction and assayed for its ability to compete with the labeled compound for binding to the selected PXR, or ligand binding domain thereof. A standard assay format employing a step to separate free known (labeled) compound from bound, or an SPA format, can be used to assess the ability of the test compound to compete.

To determine if a test compound activates a selected PXR, and thus induces P450 3A4 expression, the ligand binding domain of the selected PXR is prepared (e.g., expressed) as a fusion protein (e.g., with glutathione-S-transferase (GST), a histidine tag or a maltose binding protein). The fusion protein and coactivator (either or both advantageously labeled with a detectable label, e.g., a radiolabel or fluorescent tag) are incubated in the presence and absence of the test compound and the extent of binding of the coactivator to the fusion protein determined. The induction of interaction in the presence of the test compound is indicative of an activator of the selected PXR.

PXR activation assays in accordance with the invention can be carried out using a full length PXR and a reporter system comprising one or more copies of the DNA binding site recognized by the PXR binding domain. More preferably, however, the activation assays are conducted using established chimeric receptor systems. For example, the ligand binding domain of a selected PXR can be fused to the DNA binding domain of, for example, yeast transcription factor GAL4, or that of the estrogen or glucocorticoid receptor. An expression vector for the chimera (e.g.,a GAL4-PXR chimera) can be transfected into host cells (e.g., CV-1, HuH7, HepG2 or Caco2 cells) together with a reported construct. The reporter construct may comprise one or more (e.g., 5) copies of the DNA binding site recognized by the binding domain present in the chimera (e.g., the GAL4 DNA binding site) driving expression of a reporter gene (e.g., CAT, SPAP or luciferase). Cells containing the constructs are then treated with either vehicle alone or vehicle containing test compound, and the level of expression of the reporter gene determined. In accordance with this assay, enhancement of expression of the reporter gene in the presence of the test compound indicates that the test compound activates the selected PXR and thus can function as an inducer of CYP3A4 gene expression in that species.

Another format suitable for use in connection with the present invention is the yeast two-hybrid assay. This is an established approach to detect protein-protein interactions that is performed in yeast. Protein #1, representing the bait, is expressed in yeast as a chimera with a DNA binding domain (e.g., GAL4). Protein #2, representing the predator, is expressed in the same yeast cell as a chimera with a strong transcriptional activation domain. The interaction of bait and predator results in the activation of a reporter gene (e.g., luciferase or β-galactosidase) or the regulation of a selectable marker (e.g., LEU2 gene). This approach can be used as a screen to detect, for example, ligand-dependent interactions between a selected PXR and other proteins such as coactivator proteins (e.g., SRCI, TIFI, TIF2, ACTR) or fragments thereof (Fields et al., Nature 340:245-2.46 (1989)).

Still another format is the ligand-induced complex formation (LIC) assay. This assay detects ligand-mediated effects on nuclear receptor-DNA interactions. A selected PXR (or, minimally, DNA and/or ligand binding domains thereof) can be incubated with its heterodimeric partner RXR in the presence of DNA representing an established PXR/RXR binding site. Test-compounds can be assayed for their ability to either enhance or interfere with binding of the PXR/RXR heterodimer to DNA (Forman et al, Proc. Natl. Acad. Sci. USA 94:4312-4317 (1997).

In a preferred embodiment, the screening assay takes the form of a FRET (Fluorescence Resonance Emission Transfer assay (Nichols et al. (1998) Anal. Biochem. 257:112-119). This screening assay comprises the steps of exposing a sample portion comprising the donor located at a first position and the acceptor located at the second position to light at a first wavelength capable of inducing a first electronic transition in the donor. The donor comprises a complex of a lanthanide chelate and a lanthanide capable of binding the chelate. The spectral overlap of the donor emission and acceptor absorption is sufficient to enable energy transfer from the donor to the acceptor as measured by a detectable increase in acceptor luminescence. In a preferred embodiment, a SRC-1 (LCD2, 677-696) lanthanide chelate is used. Preferably, the lanthanide element comprises Europium and the signal chelate comprises Europium bound to a PXR of the present invention. A signal pair comprising Europium bound to the PXR and APC (allophycocyanin) bound to SRC-1 (see, e.g., Parks et al. (1999) Science 284:1365-1368) can also be used.

Suitable test compounds which can be screened in the above assays include combinatorial libraries, defined chemical entities and compounds, peptide and peptide mimetics, oligonucleotides, natural product libraries such as display libraries (e.g. phage display libraries), and antibody products.

Typically, organic molecules, preferably small organic molecules which have a molecular weight of from 50 to 2500 daltons, are screened. Candidate test compounds can be biomolecules including, saccharides, fatty acids, steroids, purines, pyrimidines, derivatives, structural analogs and combinations thereof. Such test compounds are obtained from a wide variety of sources including libraries of synthetic and natural compounds. Further, known pharmacological agents can be subjected to directed or random chemical modifications, such as acylation, alkylation, esterification, amidification, etc. to produce structural analogs.

Test compounds can be used in an initial screen of, for example, 10 compounds per reaction, and the compounds of these batches which show inhibition or activation re-screened individually. Test compounds may be screened at a concentration of from 1 nM to 1000 μM, preferably from 1 μM to 100 μM, more preferably from 1 μM to 10 μM. Preferably, the activity of a test compound is compared to the activity shown by a known activator or inhibitor. A test compound which acts as an inhibitor preferably produces a 50% inhibition of activity of the receptor. Alternatively a test compound which acts as an activator preferably produces 50% of the maximal activity produced using a known activator.

Comparative pharmacology involves the use of a nonhuman animal model to either predict the effects of a compound in humans, or provide a contrast to the effects of a compound in humans. Accordingly, results from assays such as described above with the PXRs of the present invention provide important information with respect to whether a compound of interest modulates the receptor similarly to or differently from the analogous human receptor.

Further, comparison of activation of human PXRs with activation of a non-human PXR of the present invention or PXRs from other nonhuman animals which are used as models in preclinical studies is useful in selection of preclinical animal models predictive of affects of a test compound on P450 3A4 in humans. In this method, in vitro activation of human PXR in the presence of a test compound is compared with in vitro activation of PXRs from various preclinical animal models, including, but not limited to the PXRs of the present invention, in the presence of the same test compound. A PXR from a preclinical animal model exhibiting similar in vitro activation to the human PXR in the presence of the test compound is indicative of the preclinical animal model being predictive of the affects of the test compound on P450 3A4 in humans.

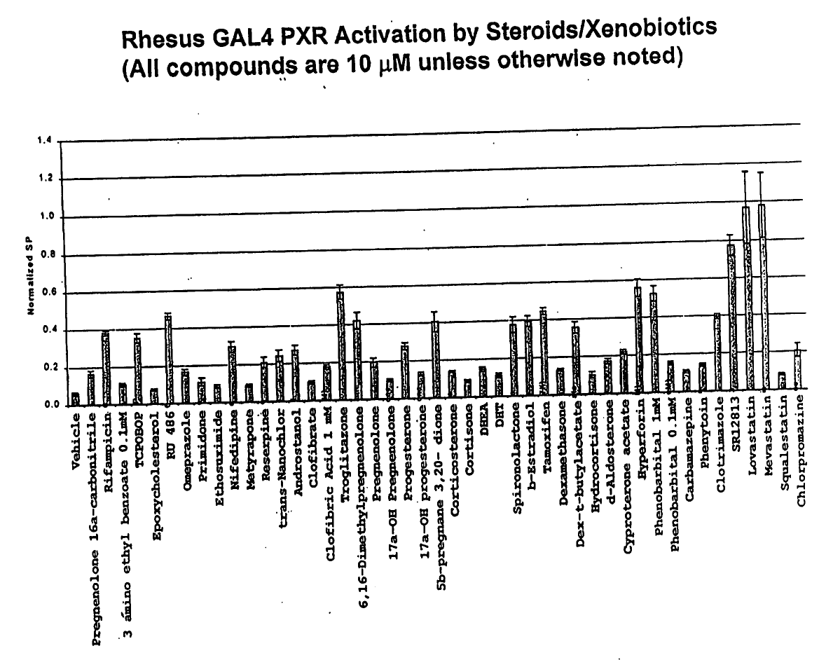

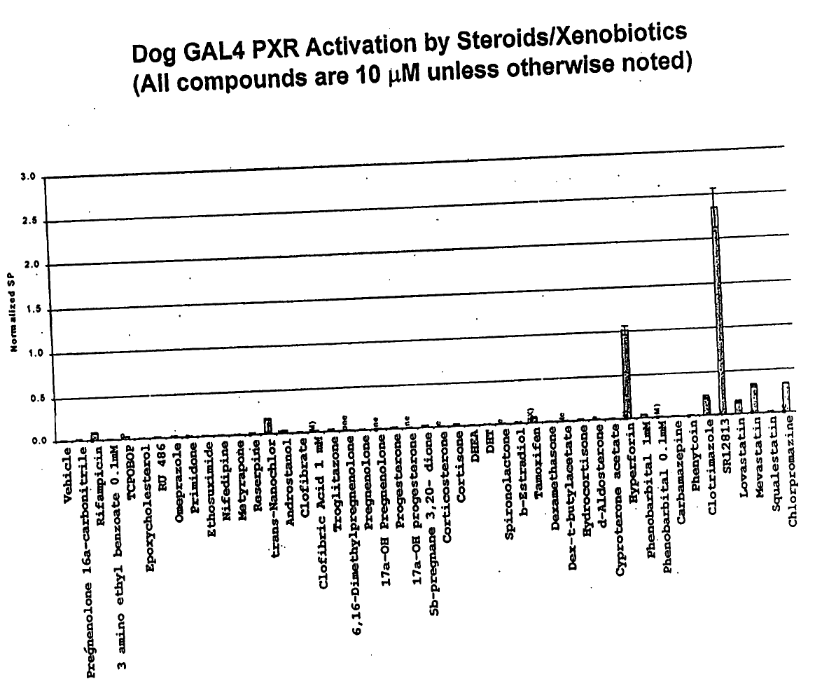

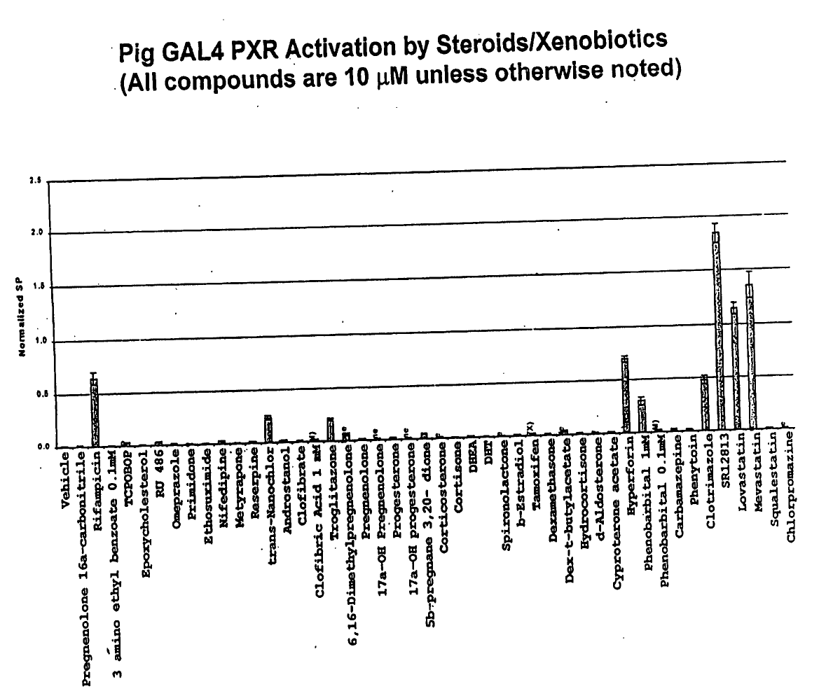

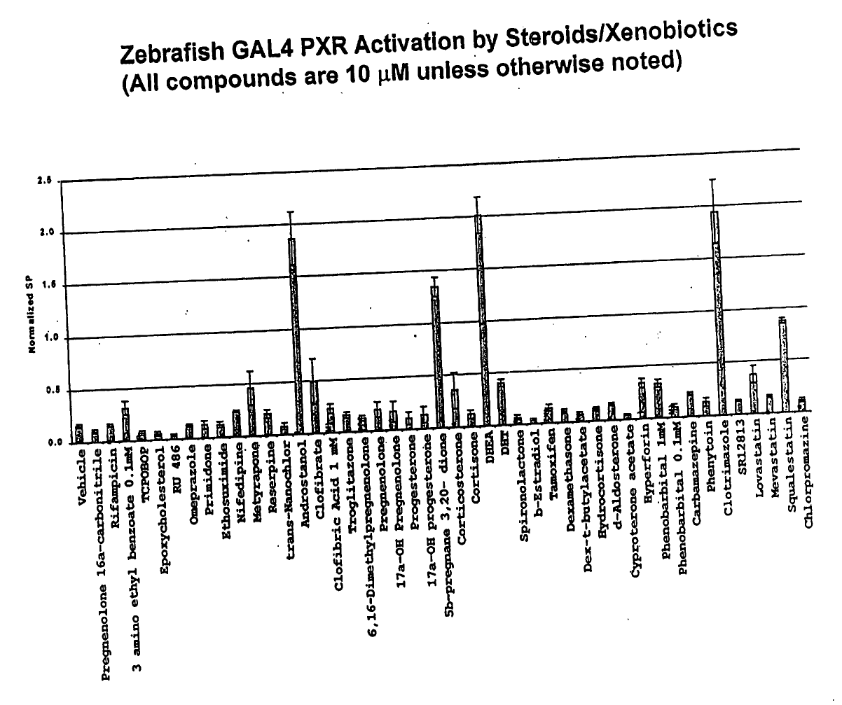

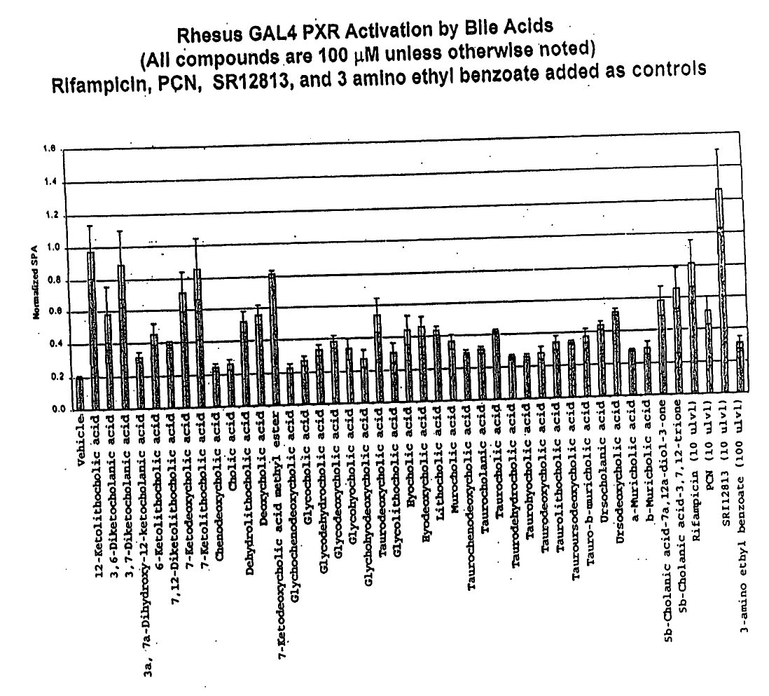

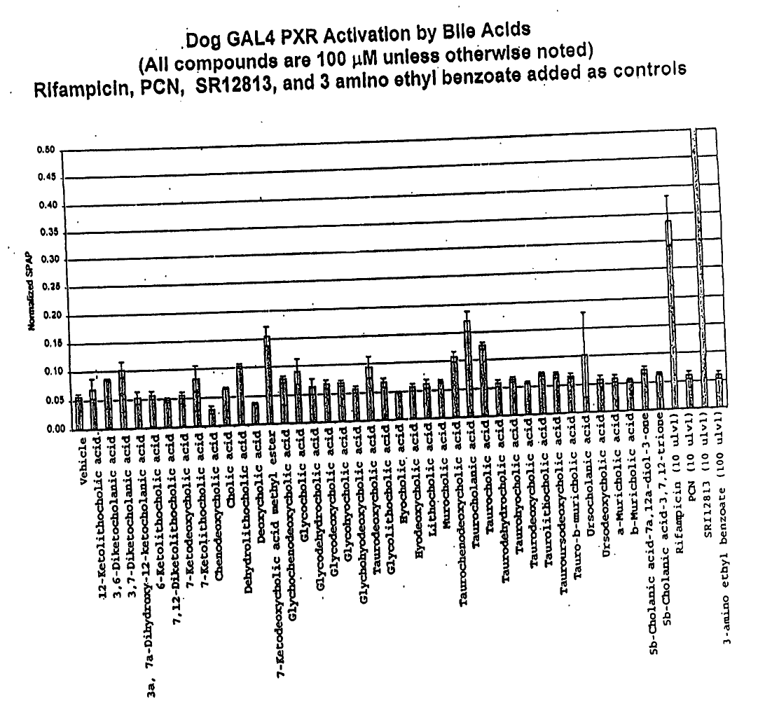

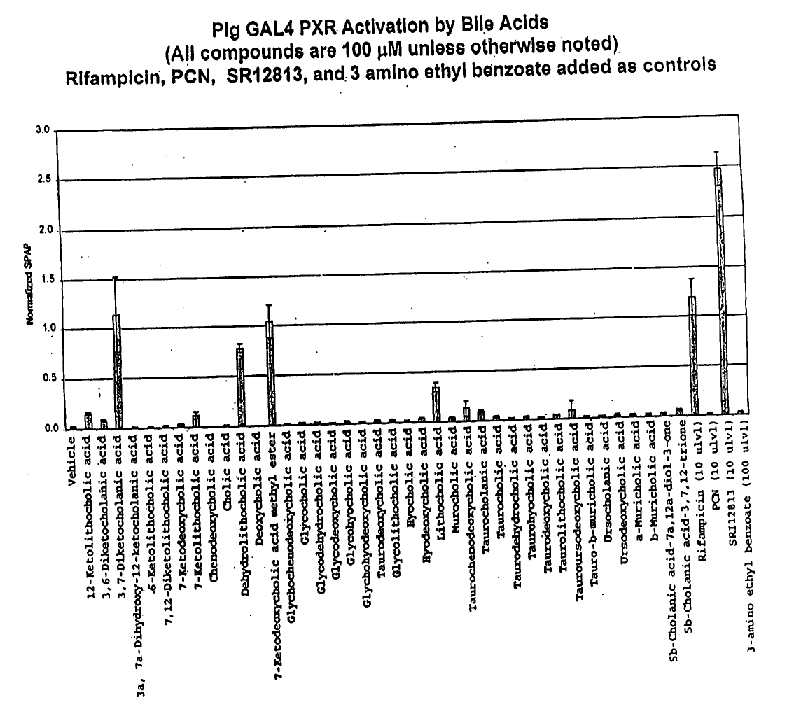

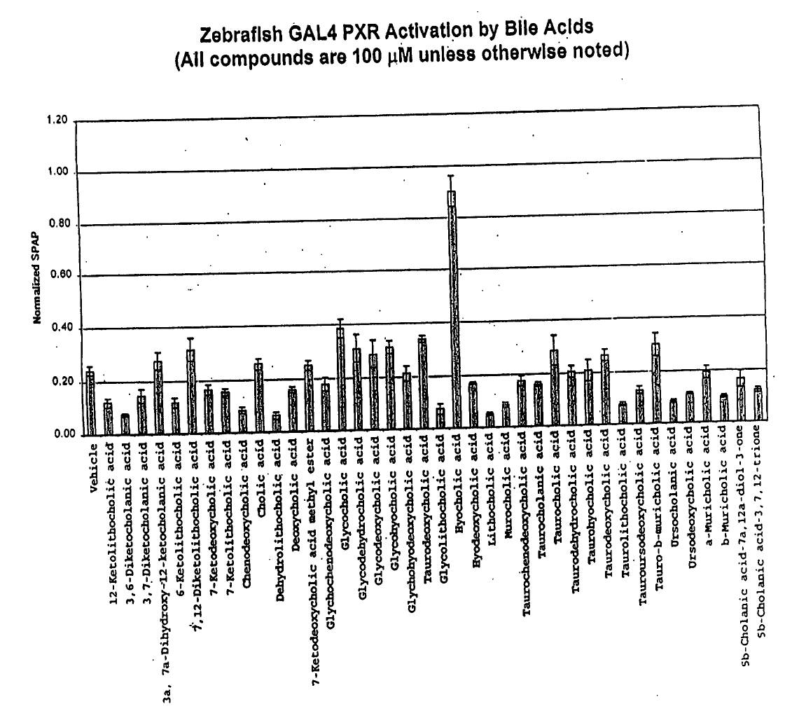

Activation of the nonhuman PXRs of the present invention was examined in the presence of various steroids, xenobiotics, and bile acids. Steroids and xenobiotics screened for effects on activation of the nonhuman PXRs of the present invention included: pregnenolone 16a-carbonitrile (PCN); rifampicine; 3 amino ethyl benzoate; TCPOBOP (1,4-bis[2-(3,5-dichloropyridyloxy)]benzene); epoxycholesterol; RU 486; omeprazole; primidone; ethosuximide; nifedipine; metyrapone; reserpine; trans-nanochlor; androstanol; clofibrate; clofibric acid; troglitazone; 6, 16-dimethylpregnenolone; pregnenolone; 17a-OH pregnenolone; progesterone; 17a-OH-progesterone 5b-pregnane 3,20-dione; corticosterone; cortisone; DHEA (dehydroepiandrosterone); DHT (dihydrotestosterone); spironolactone; b-estradiol; tamoxifen; dexamethasone; dex-t-butylacetate; hydrocortisone; d-aldosterone; cyproterone acetate; hyperforin; phenobarbital; carbamazepine; phenytoin; clotrimazole; SR12813; lovastatin; mevastatin; squalestatin; and chlorpromazine. Bile acids screened for effects on activation of the nonhuman PXRs of the present invention included: 12-ketolithocholic acid; 3,6-diketocholanic acid; 3,7-diketocholanic acid; 3a, 7a-dihydroxy-12-ketocholanic acid; 6-ketolithocholic acid; 7,12-diketolithocholic acid; 7-ketodeoxycholic acid; 7-ketolithocholic acid; chenodeoxycholic acid; cholic acid; dehydrolithocholic acid; deoxycholic acid; 7-ketodeoxycholic acid methyl ester; glychochenodeoxycholic acid; glycocholic acid; glycodehydrocholic acid; glycodeoxycholic acid; glycohyocholic acid; glychohyodeoxycholic acid; taurodeoxycholic acid; glycolithocholic acid; hyocholic acid; hyodeoxycholic acid; lithocholic acid; murocholic acid; taurochenodeoxycholic acid; taurocholanic acid; taurocholic acid; taurodehydrocholic acid; taurohyocholic acid; taurodeoxycholic acid; taurolithocholic acid; tauro-b-muricholic acid; ursocholanic acid; ursodeoxycholic acid; a-muricholic acid; b-muricholic acid; 5b-cholanic acid-7a,12a-diol-3-one; and 5b-cholanic acid-3,7,12-trione. The co-transactivation assay described in Example 2 was used to screen these compounds. Activation was assessed via measurement of levels of secreted placental alkaline phosphates normalized (normalized SPAP) to transfection levels of a control gene. Results from these experiments are depicted in FIGS. 1-8. Xenobiotics and steroids were assessed at a concentration of 10 μM unless otherwise indicated on the graph. Bile acids were assessed at a concentration of 100 μM unless otherwise indicated on the graph.

As can be seen from these experiments, PXRs from different species exhibited varying activation patterns in the presence of the same compounds. Similar activation screening assays to these can be performed with other test compounds, most preferably new drugs in development. Results from these assays are useful in PXR comparative pharmacology and selecting appropriate animal models for preclinical studies predictive of effects in humans.

The following nonlimiting examples further illustrate the present invention.

### EXAMPLES

### Example 1

### Characterization of the Sequence

Four PXR LBD sequences from pig, dog, zebrafish, and rhesus monkey were cloned. The isolation of each sequence was achieved using essentially the same strategy for each. A small stretch of the LBD was obtained using either cross-hybridizing PCR primers from another species, or by finding some portion of the LBD sequence in the EST database. The remainder of the LBD was subsequently isolated by PCR amplification of flanking sequence using a primer from within the starting sequence combined with either A) a degenerate oligo representing the canonical P-box of the DBD to isolate 5′ sequence, or B) oligo d(T)₂₀-G, d(T)₂₀-C,or d(T)₂₀-A to isolate 3′ sequence. After deriving the sequence to the poly (A) tail, the full-length LBD was produced using primers flanking the coding sequence. Wild-type sequence was determined through examination of at least three independent amplifications of each full-length LBD.

To clone pig PXR LBD, total mRNA was prepared from frozen pig liver (1g) using the FastTrack 2.0 RNA Preparation kit (InVitrogen, San Diego, Calif.). Oligo d(T)-primed cDNA synthesis was carried out by RT-PCR using a cDNA Cycle Kit (InVitrogen, San Diego, Calif.). An approximately 250 base pair stretch of pig PXR LBD was amplified from this cDNA using homologous mouse PXR LBD primers.

To clone dog PXR LBD, human PXR LBD primers were used to amplify an approximately 450 base pair fragment from a dog liver 5′-stretch λgt11 cDNA library (Clontech, Palo Alto, Calif.).

To clone rhesus PXR LBD, human primers were used to amplify all but the termini of the rhesus PXR LBD from a rhesus liver cDNA library. Primer sequences 5′-TGC CGT GTA TGT GGG GAC AAG GC-3′ (SEQ ID NO:9) and 5′-GGC ATG AAG AA GAG ATG ATC ATG-3′ (SEQ ID NO:10) were used to amplify a 274 base pair fragment from a Rhesus liver library constructed in the CMVSport6 vector. The fragment was sequenced and new primers were designed based on this Rhesus sequence. These primers were then used with vector arm primers to amplify the regions 5′ and 3′ of the known sequenced fragment.

To clone zebrafish (Danio rerio) PXR LBD, an initial fragment of the PXR LBD was identified as an EST sequence (Accession # AI943313). This sequence was used to design primers for amplification of the entire LBD from cDNA synthesized using zebrafish embryo (48h) oligo d(T)-primed cDNA.

### Example 2

### Characterization of Selected PXRs Via Cotransfection Assays

In order to assess ligands for their ability to activate PXR in a cell-based assay, a transient transfection approach was utilized. PXR ligand binding domains were fused to the Gal4 DNA binding domain and tested against a reporter gene regulated by a Gal4 response element (from the yeast UAS_{G}). Lipofectamine-based transient transfection assays were conducted as described previously (Jones et al., 2000 Molecular Endocrinology, volume 14, pp. 27-39), except that a UAS-tk-SPAP reporter vector was used instead of the (CYP3A1 DR3)₂-tk-CAT reporter vector. When testing bile acids, an expression plasmid encoding intestinal bile acid transporter (IBAT) was added to facilitate cellular uptake of bile acids.

What is claimed is:

1. An isolated pregnane X nuclear receptor polypeptide comprising:
(a) an amino acid sequence of SEQ ID NO: 2, 4, 6 or 8;
(b) a variant of the amino acid sequence as defined in (a) which modulates P450 3A4 levels or activity; or
(c) a fragment of (a) or (b) which modulates P450 3A4 levels or activity.

2. A polypeptide according to claim 1 wherein the variant (b) has at least 80% identity to the amino acid sequence of SEQ ID NO: 2, 4, 6 or 8.

3. A polynucleotide encoding a polypeptide according to claim 1.

4. A polynucleotide according to claim 3 which is a cDNA sequence.

5. A polynucleotide encoding a pregnane X receptor polypeptide which modulates P450 3A4 levels or activity, said polynucleotide comprising:
(a) a nucleic acid sequence of SEQ ID NO: 1, 3, 5 or 7;
(b) a nucleic acid sequence which hybridizes under stringent conditions to the nucleic acid sequence as defined in (a);
(c) a nucleic acid sequence that is degenerate as a result of the genetic code to the nucleic acid sequence as defined in (a) or (b); or
(d) a nucleic acid sequence having at least 60% identity to the nucleic acid sequence as defined in (a), (b) or (c).

6. The polynucleotide of claim 3 wherein the polynucleotide encodes amino acids 106 to 434 set forth in SEQ ID NO: 4.

7. The polynucleotide of claim 3 wherein the polynucleotide encodes amino acids 41 to 105 set forth in SEQ ID NO:4.

8. The polynucleotide of claim 3 wherein the polynucleotide encodes amino acids 1 to 40 set forth in SEQ ID NO:4.

9. A fusion protein comprising:
(a) a DNA binding or ligand binding domain of the pregnane X receptor of claim 1; and
(b) a non-pregnane X receptor-derived amino acid sequence.

10. An isolated polynucleotide encoding the fusion protein of claim 9.

11. An expression vector comprising a polynucleotide according to any one of claims 3 to 8 or 10.

12. A host cell comprising an expression vector according to claim 11.

13. An antibody specific for a polypeptide according to claim 1.

14. A method for the identification of a compound that modulates pregnane X receptor activity and/or expression, said method comprising:
(a) contacting a test compound with a canine, porcine, primate or Zebrafish pregnane X recpetor polypeptide or polynucleotide; and
(b) determining an effect of the test compound on the activity and/or expression of said polypeptide or polynucleotide.

15. A method according to claim 14 wherein the polypeptide is expressed in a cell.

16. A substance which modulates pregnane X receptor activity and which is identifiable by a method according to claim 14 or 15.

17. A non-human transgenic animal expressing a PXR polypeptide of claim 1 or a mutant thereof.

18. A method for selecting a preclinical animal model which is predictive of affects of a test compound on P450 3A4 in humans comprising comparing in vitro activation of human PXR in the presence of the test compound with in vitro activation of PXRs from preclinical animal models in the presence of the test compound, wherein a PXR from a preclinical animal model exhibiting similar in vitro activation to the human PXR is indicative of the preclinical animal model being predictive of the affects of the test compound on P450 3A4 in humans.

19. The method of claim 18 wherein the pregnane X receptor from the preclinical model is a canine, porcine, primate or Zebrafish pregnane X receptor.


SEQUENCE LISTING (8 sequences)

SEQ ID NO: 1 - DNA, 990 bp, Canine
  CDS (1)...(990)
ggcatgaagaaggagatgatcatgtccgacgcggctgtggagcagaggcgggctctgatccggaggaaaaagcgagaacggatgggcgcgtcgccgctgggagccaaggggctgagtgaggagcagcagacgatgatccgagagctgatggatgcccagatgaaaacctttgacaccaccttctccaacttcaaggatttccggctgccggccgcgtgcagcagcgggcgcgaggtcccaggagcggcgcacactccagtgggggaggaagctgccaagtggagccaggtcagggaggatctgtgctcgctgaaggtgtgcctgcggctgcgcggggaggacggcagcgtccagaactacacaccccaggccgaccgcagcggcgccgagatcttttccctgctgccccacatggctgacatgtccacctacatgttcaaaggcgtcatcaactttgccaaagtcatctcccacttcagggaattgcccatcgaggaccagatctcgctgctaaagggggccaccttcgaggtgtgccagctgaggttcaacacggtgttcaacgcagagaccggaacctgggagtgtggccggctgtcctactgcctggaagaccctgcaggcggcttccagcagcttctcctggagccagtgctgaagttccactacaggctgaagaggctgcagctgcataaggaggagtatgtgctgatgcaggccatctctcttttctccccagaccgcccaggtgtggtgcagcgcagcgtggtggaccagctgcaggagagatttgccatcgccctgaaggcctacatcgagtgcaatcggccgcagcctgcccaccggttcctgttcctgaagatcatggccatgctcaccgagcttcgcagcatcaatgcccagcacacccagaagctgctgcgcatccaggacatacaccccttcgccagccccctcatgcaggagctgttcagcatcacggacggctga

SEQ ID NO: 2 - protein, 329 aa, Canine
GMKKEMIMSDAAVEQRRALIRRKKRERMGASPLGAKGLSEEQQTMIRELMDAQMKTFDTTFSNFKDFRLPAACSSGREVPGAAHTPVGEEAAKWSQVREDLCSLKVCLRLRGEDGSVQNYTPQADRSGAEIFSLLPHMADMSTYMFKGVINFAKVISHFRELPIEDQISLLKGATFEVCQLRFNTVFNAETGTWECGRLSYCLEDPAGGFQQLLLEPVLKFHYRLKRLQLHKEEYVLMQAISLFSPDRPGVVQRSVVDQLQERFAIALKAYIECNRPQPAHRFLFLKIMAMLTELRSINAQHTQKLLRIQDIHPFASPLMQELFSITDG

SEQ ID NO: 3 - DNA, 1441 bp, Primate PXR
  CDS (119)...(1423)
tccttggtaaagctactccttgatcgatcctttgcacctgattgttcaaagtggaccccagggggaagtcagagcgaagaacttaccgctaagcagtccaagaggcccagaagcaaacctggaggtgagacccaaagaaggctggaaccatgctgactttgtatactgtgaggacacagagtttgctcctggaaagcccactgtcaacgcagatgaggaagttgggggtccccaaatctgccgtgtatgtggggacaaggccactggttatcacttcaatgtcatgacatgtgaaggatgcaagggctttttcaggagggccatgaaacgcaacgcccgccttaggtgccccttccggaagggcgcctgcgagatcacccggaagacccggcgacagtgccaggcctgccggctgcgcaagtgcctggagagcggcatgaagaaggagatgatcatgtccgacgcggccgtagaggagaggcgggccttgatcaagaggaagaaaagagaacggatcgggactcagccacccggagtgcaggggctgacggaggagcagcggatgatgatcagggagctgatggacgctcagatgaaaacctttgacactaccttctcccatttcaagaatttccggctgccaggggtgcttagcagtggctgtgagatgccagagtctctgcaggccccatcgagggaagaagctgccaagtggaaccaggtcaggaaagatctgtggtctgtgaaggtctccgtgcagctgcggggggaggatggcagtgtctggaactacaaacccccagccgacaatggcgggaaagagatcttctccttgctgccccacatggctgacatgtcaacctacatgttcaaaggcatcatcaactttgccaaagtcatctcctacttcagggacctgcccatcgaggaccagatctccctactgaagggggccacttttgagctgtgccagctgagattcaacacagtattcaacgcggagactggaacttgggagtgtggccggctgtcctactgcttggaagaccctgcaggtggtttccagcaacttctgctggagcccatgctgaaattccactacatgctgaagaagctgcagctacacgaggaggagtatgtgctgatgcaggccatctccctcttctccccagaccgcccaggtgtggtgcagcaccgcgtggtggaccagctgcaggagcaatacgctattactctgaagtcctacattgaatgcaatcggccccagcctgctcataggttcctgttcctgaagatcatggctatgctcaccgagctccgcagcatcaacgcccagcacacccagcggctgctgcgcatccaggacatacacccctttgctacgcccctcatgcaggagttgttcggcatcacgggtagctgagtggctgcccttgggtga

SEQ ID NO: 4 - protein, 434 aa, Primate PXR
LEVRPKEGWNHADFVYCEDTEFAPGKPTVNADEEVGGPQICRVCGDKATGYHFNVMTCEGCKGFFRRAMKRNARLRCPFRKGACEITRKTRRQCQACRLRKCLESGMKKEMIMSDAAVEERRALIKRKKRERIGTQPPGVQGLTEEQRMMIRELMDAQMKTFDTTFSHFKNFRLPGVLSSGCEMPESLQAPSREEAAKWNQVRKDLWSVKVSVQLRGEDGSVWNYKPPADNGGKEIFSLLPHMADMSTYMFKGIINFAKVISYFRDLPIEDQISLLKGATFELCQLRFNTVFNAETGTWECGRLSYCLEDPAGGFQQLLLEPMLKFHYMLKKLQLHEEEYVLMQAISLFSPDRPGVVQHRVVDQLQEQYAITLKSYIECNRPQPAHRFLFLKIMAMLTELRSINAQHTQRLLRIQDIHPFATPLMQELFGITGS

SEQ ID NO: 5 - DNA, 993 bp, Porcine PXR
  CDS (1)...(993)
ggcatgaggaaggaaatgatcatgtcagatgcagctgtggagcagaggcgggccttgatcaggaggaagaaacgagaacagatcggggctcagcccccaggagccaagggtctcactgaagagcagcggacaatgatcagtgagctgatgaacgctcagatgaaaacctttgacaccaccttcacacatttcaagaattttcggttaccagaggtgcttagcagtagcctcgagattccagagtgtctgcagactccgtcgtcaagggaagaagctgccaagtggagcaagctcagggaagatctgtgctcagtgaaactctctctgcagctaaggggggaagatggtagcgtctggaactacaaacccccagcagacaacagtgggaaagagatcttttccctgctgccccacatagctgacatgtcaacctacatgttcaaaggcattatcaactttgccaaagtcatctcctacttcagggacttgcccattgaggaccagatctctctgctgaagggggccacctttgagctgtgccagctgagattcaacacggtgttcaacgcagagacggggacctgggagtgtggtcggctgtcctacagcttggaagacccctcaggtggcttccagcagcttctcctgcagcccatgctgaaattccactacatgctgaagaagctgcagctgcataaggaggagtatgtgctgatgcaggccatctcccttttctctccagaccgcccgggtgtggtgcaacgccaagtggtggaccagctgcaggagaggtttgccattaccctgaaggcctacatcgagtgcaaccggccccagcctgcccaccgattcctgttcctgaagatcatggctatgctcactgagctccgcagcatcaacgcccaacacacccagcggctgctgcgaatccaggacatacaccccttcgccaccccactcatgcaggagttattcagcatcacagaaagctga

SEQ ID NO: 6 - protein, 330 aa, Porcine
GMRKEMIMSDAAVEQRRALIRRKKREQIGAQPPGAKGLTEEQRTMISELMNAQMKTFDTTFTHFKNFRLPEVLSSSLEIPECLQTPSSREEAAKWSKLREDLCSVKLSLQLRGEDGSVWNYKPPADNSGKEIFSLLPHIADMSTYMFKGIINFAKVISYFRDLPIEDQISLLKGATFELCQLRFNTVFNAETGTWECGRLSYSLEDPSGGFQQLLLQPMLKFHYMLKKLQLHKEEYVLMQAISLFSPDRPGVVQRQVVDQLQERFAITLKAYIECNRPQPAHRFLFLKIMAMLTELRSINAQHTQRLLRIQDIHPFATPLMQELFSITES

SEQ ID NO: 7 - DNA, 1008 bp, Zebrafish
  CDS (1)...(969)
ggcatgaagagagagctgatcatgtcggatgaggcggtggagaagcggaggttgcagatcaggaggaagaggatgcaggaagagcctgtaactctcactcctcaacaggaagctgtcatacaagagctgctgaacgcacacaagaaaaccttcgacatgacttgtgcccatttcagtcagttccggcctttagatcgggatcagaagtctgtgtccgagtcgagtccactcacaaacggcagctggatcgatcacagacccatcgctgaagacccaatgcagtgggtcttcaatcccacttcgctctcgtcctcttcctccagctaccagagccttgacaataaagagaagaagcactttaaaagtggcaacttctcctctctgccacacttcacagacctcaccacgtacatgatcaagaatgtcatcaacttcgggaagacgctgacaatgtttagggctctggttatggaggaccagatctcgctgctgaaaggtgccacctttgagatcattctgattcacttcaacatgttctttaatgaagtgacgggaatttgggagtgcggccccctgcagtactgcatggatgatgcctttcgagctggttttcagcaccatctgctggacccaatgatgaatttccattacacactgcgtaagctgcgtttgcatgaggaggagtatgtgctgatgcaggccctctctctcttttcaccagatcgccctggtgtgacagaccacaaagtgatcgaccgcaaccaggaaacactagcgcttaccctaaagacttacattgaggccaagagaaatgggccagaaaaacatctgctgttcccaaagattatggggtgcctgaccgagatgaggagcatgaacgaagagtacaccaaacaagtgctgaaaatccaggacatgcagcctgaagtgtctccactttggttggaaataataagcaaagacacctaagtcctcacaaagcacgtgcaactctacttattcccaact

SEQ ID NO: 8 - protein, 322 aa, Zebrafish
GMKRELIMSDEAVEKRRLQIRRKRMQEEPVTLTPQQEAVIQELLNAHKKTFDMTCAHFSQFRPLDRDQKSVSESSPLTNGSWIDHRPIAEDPMQWVFNPTSLSSSSSSYQSLDNKEKKHFKSGNFSSLPHFTDLTTYMIKNVINFGKTLTMFRALVMEDQISLLKGATFEIILIHFNMFFNEVTGIWECGPLQYCMDDAFRAGFQHHLLDPMMNFHYTLRKLRLHEEEYVLMQALSLFSPDRPGVTDHKVIDRNQETLALTLKTYIEAKRNGPEKHLLFPKIMGCLTEMRSMNEEYTKQVLKIQDMQPEVSPLWLEIISKDT